(19) United States
(12) Patent Application Publication
(10) Pub. No.: US 2004/0127838 A1
(43) Pub. Date: Jul. 1, 2004

(54) Article, system, and method of covering and protecting portions of human skin

(75) Inventor: Michael C. Jeziak, Roseville, MI (US)

Correspondence Address: DOBRUSIN & THENNISCH PC, 401 S OLD WOODWARD AVE, SUITE 311, BIRMINGHAM, MI, 48009, US

(73) Assignee: Inkslingers, Inc., Roseville, MI

(21) Appl. No.: 10/642,911

(22) Filed: Aug. 18, 2003

Related U.S. Application Data
(60) Provisional application No. 60/404,066, filed on Aug. 16, 2002.

(51) Int. Cl. A61F 13/00

(52) U.S. Cl. 602/43

(57) ABSTRACT

An improved system, method and kit for the protection of newly applied body art. In one aspect, a system is providing having a cover adapted to create a gapped region between the newly applied body art and the cover thereby preventing unwanted contact with the same. The system also contemplates a retention member for affixing the cover to the surrounding skin or otherwise. In another aspect, the present invention also contemplates the method of creating the gapped region for the protection of a newly applied body art. In yet another aspect, the present invention further contemplates a kit comprising a cover and a retention member for the protection of a newly applied body art.

### CLAIM OF BENEFIT OF FILING DATE

The present application claims the benefit of the filing date of U.S. Provisional Application Serial No. 60/404,066 (filed Aug. 16, 2002), hereby incorporated by reference.

### FIELD OF THE INVENTION

The present invention relates generally to improved devices, systems, processes and methods for the care, protection, and coverage of areas of human skin and flesh after body art (such as a tattoo, piercing, scaring, insertion of foreign objects, removal of skin, otherwise or combinations thereof) has been applied to the human skin. More particularly, the present invention relates to a method for selecting and applying a cover, guard, shelter or shield over an area of human skin or flesh that surrounds a body art or tattooed area of the human skin immediately after the tattoo has been applied to avoid inadvertent contact to the tattooed area to facilitate proper healing and permanent placement of the selected tattoo or body art on the human skin or dermal layer. It is contemplated that the present invention relates to the placement and retention of a protective device immediately over a newly-tattooed area of human skin which allows the wearer or user to work, sleep, exercise, bathe, shower, or otherwise engage in daily activities without the risking of accidental or inadvertent touching, bumping, or trauma to the tattooed area while the newly-tattooed area heals and becomes permanent.

### BACKGROUND OF THE INVENTION

Traditional tattoo and body art application procedures involve the use of indelible and ink-like materials being fixedly administered to human skin through needle application. For example, U.S. Pat. No. 6,352,546 to Hill, hereby incorporated by reference, discusses a traditional tattoo application process wherein a needle is used to penetrate the skin which leaves a small amount of ink resulting in targeted discoloration of the selected portion of human skin. Repeating this procedure in a particular desired pattern or color scheme with different colors of ink leads to the creation of a desired tattoo. Depending upon the physical placement and design complexity of a given tattoo, it is possible that a needle may be inserted into the human skin or dermal layer thousands of times to achieve the desired tattoo or other selected form of body art. In addition to causing physical discomfort when the needle is inserted, the tattoo process also requires a period of healing immediately following the application of the tattoo or other selected piece of body art wherein the areas where the needle was inserted can heal and the ink placed therein can become permanently affixed.

During this healing period which can take up to a period of days depending upon the individual person, the area of tattoo coverage, and the portion of the body tattooed, the newly-tattooed individual can experience a certain degree of pain and discomfort if the tattooed area is accidentally or inadvertently touched, rubbed, or bumped by an outside object. In addition to physical discomfort, interference or touching of the freshly tattooed area can result in the smearing or other juxtaposition of the tattoo and diminish the clarity and color of the tattoo. Further, trauma to the newly tattooed area could cause physical scarring to the skin in the tattooed area and lead to an unattractive tattoo and scarred human skin. Still further, an individual who has a tattoo applied to his or her skin needs to refrain from swimming or otherwise having water or sunlight come into contact with the tattooed area until the healing process is complete and the tattoo permanently sets in its desired position.

Accordingly, there is need for a simple and low cost article and method whereby tattoo and body art customers can utilize a safe and hygienic cover or guard for placement over the area of tattooed skin to prevent accidental or inadvertent touching or trauma to the tattooed area while the tattoo area heals and the tattoo itself permanent affixes to the selected area of human skin. The cover, guard, or shield can be used be wearers while driving, working, or otherwise engaging in physical movement. The cover or guard can be attached or mounted over the desired area of human skin and yet may be removable by the wearer. In addition, the cover can be waterproof to allow the wearer the swim, shower, or bathe as well as tinted or colored to prevent the exposure of the tattooed area to sunlight.

### SUMMARY OF THE INVENTION

In one aspect, the present invention provides a system adapted to protect a newly applied body art. The system comprises a cover having a peripheral portion and a mating surface configured for placement on a skin surface and is shaped to surround newly applied body art on the skin surface. The cover also has a central portion joined with the peripheral portion, the central portion having an outer surface and an interior recessed surface. The system further comprises a retention member adapted to affix the cover to the skin such that placement and affixing of the cover around and over the newly applied body art creates a gapped region between the central portion and the newly applied body art and substantially prevents destructive contact (e.g. smearing, smudging, scarring, otherwise or combinations thereof) to the same. The gapped region also prevents physical discomfort or trauma to the skin region having the body art.

In another aspect, the present invention provides a method of protecting a newly applied tattoo comprising the steps of

i) providing a skin surface having a newly applied tattoo;

ii) placing a cover over the newly applied body art, the cover defined by:

a peripheral portion having a mating surface for placement on a skin surface, wherein the mating surface is shaped to surround newly applied body art on the skin surface,

a central portion joining the peripheral portion, wherein the central portion has an outer surface and an interior recessed surface;

iii) affixing the cover to the skin using a retention member, wherein a gapped region is created between the central portion and the newly applied body art, which substantially prevents destructive contact to the newly applied body art.

In yet another aspect, the present invention provides a kit for protecting a newly applied body art. The kit comprises a dome shield, which is adapted to cover at least a portion of a newly applied body art on a skin surface. Preferably, the dome shield has a recessed interior surface. The kit also comprises a retention member for affixing the shield to a skin portion.

### DESCRIPTION OF THE DRAWINGS

FIG. 1 is a top view of the system of the present invention;

FIG. 2 is a cross section taken through FIG. 1.

### DETAILED DESCRIPTION OF THE INVENTION

The present invention is directed to an article, system and method of applying a cover, guard, or shield over an area of human skin that has recently been tattooed or otherwise received an application of body art. It is contemplated that the present invention would be used by body art customers or recipients immediately after the tattoo application process, or otherwise, and provides a flexible cover to protect or shield the tattooed area from accidental or inadvertent contact with an outside force or object.

The flexible cover can be retained or secured to a selected portion of the human anatomy adjacent to the tattooed area by a retention member or plurality of retention members known in the art, such as tape, Velcro, elastic, bandages, medicated bandages, gels, etc. More particularly, the present invention allows a user to work, sleep, exercise, bathe, shower, or otherwise engage in daily activities without the risking of accidental or inadvertent touching, bumping, or trauma to the tattooed area while the newly-tattooed area heals and becomes permanent.

The protection afforded by the present invention may serve to further include a waterproof area and a sun-block feature to prevent foreign material and/or from coming into contact with the tattooed area. By providing a cover disposed over the tattooed area, the present invention may reduce the likelihood that objects or material will come into contact with the affected human skin thereby reducing potential pain and discomfort felt by the user (i.e. when the cover “blocks” or “impedes” the object from coming into direct contact with the tattooed area) and allows the tattoo to fully set and place into the area without smearing, smudging, or creating any unwanted scarring to the human skin. Since unwanted objects and matter is blocked or shielded from coming into contact with the tattooed area during the healing process, the possibility of infection, scarring, unwanted insect or animal bites, or obscuring of the tattoo design or artwork is reduced while the area of affected skin is allowed to properly heal and the selected tattoo is allowed to become permanently affixed to the skin.

Further, it is contemplated that the present invention would not be limited to use by individuals seeking to cover or guard or recent body piercing or tattooed area. In this regard, the present invention may be utilized for covering and protecting other types of wounds, punctures, or abrasions to human skin to facilitate medical treatment and healing, such as those caused by traditional injuries, burns, scraps, and other wounds relating to pain and/or discomfort to a dermal layer of a human.

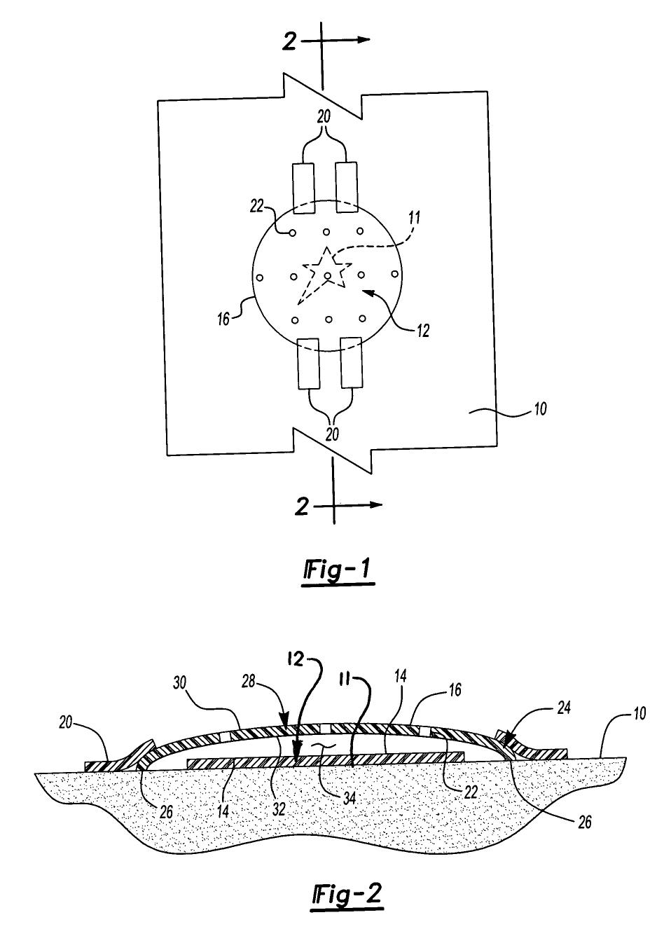

Referring to FIGS. 1 and 2, the system, device, or article of the present invention generally employs a kit or combination of materials for application to a dermal layer or area of human skin 10, which has recently received a tattoo or other application of body art. It is contemplated that a specific tattoo or other body art 11 is selected by the customer for application to an area of human skin 10. The selected tattoo or body art 11 is then applied or placed to a defined portion or area 12 of the human skin 10, which corresponds to the display pattern, or design. The method of application of the tattoo or body art is performed through methods and systems that are known in the art. Further, as known in the art, the selected tattoo or body art 11 can be placed onto any part or portion of the human anatomy and may comprise any shape or geometric design which flows along the contours of the selected human skin 10, human appendage, or other body part.

After placement of the selected tattoo or body art 11 on the skin 10 of the user, an optional layer of wound protecting device 14 or otherwise may be placed over the body art for protection. Examples of such devices includes gauze, medicinal layer, bandage or otherwise, which can then be placed directly over the tattooed area of the defined portion or area 12 of human skin 10 to facilitate healing of the tattooed area or otherwise absorb blood and other residue resulting from the multiple needle penetrations typically encountered in the application of a conventional tattoo. Likewise, it is contemplated that agents (e.g. ointments, gels, lotions, or otherwise) may be alternatively or in conjunction with the wound protection device 14 to further facilitate in the healing process.

A covering member 16 is then placed over the entire surface area of the defined portion or area 12 of the skin that received the tattoo 11 thereby covering the tattooed area. The cover of the present invention is configured with a peripheral portion 24 around the edge of member 16. The peripheral portion has a mating surface 26 configured for placement on a skin surface area 12 and is shaped to surround newly applied body art 11 on the skin surface area 12. The cover 16 also has a central portion 28 joined with the peripheral portion 24, the central portion 28 has an outer surface 30 and an interior recessed surface 32. As such, when the mating surface 26 is placed around and over the newly applied body art 11 a gapped region 34 between the central portion 28 and the newly applied body art to skin region 12 is created, which substantially prevents destructive contact to the newly applied body art 11.

The cover member 16 may be configured such that placement of the cover 16 onto the surface of the skin 10 creates a substantially waterproof environment in the gapped region. As such, the peripheral portion 24 and central portion 28 of the cover member 16 are configured without openings. Alternatively, the cover or member 16 may have a plurality of perforations 22 extended through both the inner or interior portion and the outer or exterior portion of the member 16 which allows air to flow over the human skin 10 and allows heat and human sweat to discharge from the volume or gapped region 34. In addition, it is contemplated that the user may utilize the perforations 22 as a port to place medication directly into contact with the skin 10, particularly if wound-protecting device 14 (e.g. bandage or otherwise) is present.

The cover or member 16 can be rigid or flexible and preferably consists of a metal, metal alloy, nylon, an epoxy-based resin, or a plastic such as polyethylene, polypropylene, or polystyrene and may be formed through injection molding or reaction injection molding processes which are well known in the art. The cover or member 16 may be tinted, opaque, or darkened to reduce sun exposure to the tattooed area while the skin 10 is undergoing the healing process and is defined as having an outer or exterior portion which is visible to the user and an inner or interior portion which is placed over the tattooed area of the human skin 10.

Generally speaking, the cover or member 16 can be of any geometric shape or configuration, which corresponds to the shape or design of the selected tattoo. Namely, the cover or member 16 can guard, shield or shelter a body art 11 placed upon the skin area 12 or comprise a larger member 16 for covering a large display or shield a piece of body art attached to the skin 10. More particularly, the member or cover 16 can comprise any shape or configuration which defines an area or surface area disposed over human skin or other area of human anatomy. Further, the cover or member 16 also comprises a height element, which defines a volumetric area. This volume can be achieved through the use of a variety of designs, shapes, or configurations that are well known in the art and can even be customized for aesthetic or design effect, such as a dome, moon-shape, or other configuration having any number of height parameters and creating a wide variety of spacing between the human skin 10 and the inner or interior portion of the cover or member 16.

Although the member or cover 16 can be of any shape or configuration to cover the area defined by a tattoo, it is generally convex or dome-like in height thereby defining the gapped region 34. For example, an air pocket may be formed, which is defined between the skin area 12 and the recessed surface 32 of the central portion 28. When an optional adapted to facilitate in the healing process of the newly applied body art is applied over the area or portion 12 of the skin 10, the gapped region 34 is defined between the wound-protecting device 14 and the recessed interior surface 32 of the central portion 28. For example, the cover or member 16 can comprise a back-brace type of configuration for tattoos disposed on the back of a user which would allow the user to operate a motor vehicle without having the tattooed area come into contact with the seat of the motor vehicle since the gapped region 34 between the member 16 and the skin 10 would serve as a barrier, guard, or shield.

The cover or member 16 is retained or secured to the skin through a retention member 20 or plurality of retention members 20 such as a tape, Velcro, adhesive, bandage, gauze, wrap, and the like. It is contemplated that the retention member or members 20 of the present invention may be selected from any type of material or mechanical fastening system used to effectuate the securing and retention of the cover or member 16 in the desired location over the human skin 10 and prevent the member 16 from moving or otherwise passing over the human skin 10. In an alternative embodiment, the retention member 20 may have a waterproof feature whereby the ingress or egress of water through the cover or member 16 is prevented or reduced thereby allowing the user to bathe, swim, exercise, or take a shower without allowing water to come into contact with the tattooed area.

The present invention further serves to provide a method or process for applying the cover or member 16 over the human skin 10 to serve as a shield or guard to ensure that unwanted objects and materials do not come into contact with the newly-applied tattoo 11 which could obscure the tattoo or cause discomfort, pain, or infection of the portion or area 12 of the human skin 10 where the tattoo or body art 11 was applied. The specific objects, specifications, features, and improvements of the present invention can be briefly summarized as part of the following steps:

1. After application of a conventional tattoo or other piece of body art, the area of skin where the tattoo is applied is often sore and prone to discharge due to the large number of needle penetrations required to create the selected tattoo design 11 on the user. Depending upon the individual, the selected tattoo design 11, and the area of the body 12, a healing process of a matter of hours or days is typically required before the tattoo permanently sets and the user does not experience any physical discomfort. During this healing process and time period, inadvertent or unwanted touching of the area can lead to the smearing, smudging, or otherwise obscuring of the tattoo design 11 and a painful, unpleasant, or uncomfortable sensation to the user. Moreover, the user may need to restrict certain types of activities during this period such as swimming, exercise, or placing pressure on the tattooed area.

2. The method of the present invention addresses these issues by providing process and steps of first, optionally applying a wound-protecting device 14 (e.g. bandage or otherwise) or an agent adapted to facilitate in the healing process of the newly applied body art (e.g. medicated cream, ointment, Vasiline™, lotion or otherwise).

3. A convex or dome-like cover member 16 corresponding to the shape, geometry, or dimensions of the user's tattoo 11 is then placed over the tattooed area

12. The convex or dome-like volumetric dimension of the cover, member, or covering member 16 creates a gapped region 34 (e.g. an air pocket) whereby the tattooed area 12 is separated from an inner or interior portion of the cover or member 16. The cover or member 16 is then secured or retained to human skin surrounding the tattooed area by tape or other adhesive or retention means 20 which fixedly places the cover or member 16 over the tattooed area 12 and allows the cover 16 to act as a guard or shield to prevent unwanted contact with outside objects. The cover or member 16 can be of any shape or color and can have additional properties to block sunlight from reaching the tattooed area.

4. It is possible that the cover can be retained to the human skin in a waterproof or watertight condition, which would allow the user to swim, shower, or bathe without allowing water to come into contact with the tattooed area. In the alternative, the cover may have a plurality of perforations, which allows the tattooed area to breath or allow the introduction of additional cream or ointment to be placed into contact with the tattooed area.

Furthermore, the method contemplates any other aspect of the present invention disclosed herein.

In yet another aspect, the present invention provides a kit for protecting a newly applied body art. The kit comprises a dome shield 16, which is adapted to cover at least a portion of a newly applied body art 11 on a skin surface 10. Preferably, the dome shield 16 has a recessed interior surface 32. The kit also comprises a retention member 20 for affixing the dome shield 16 to a skin portion 10.

Additionally, the kit may further comprise of a wound protecting device 14 or an agent adapted to facilitate in the healing process of the newly applied body art.

In another embodiment, the present invention may comprise a flexible sheet material having adjustable sizing or stretch releasing characteristics such as those disclosed in U.S. Pat. No. 6,572,945, which is hereby incorporated by reference. In this embodiment, the cover may consist of a sheet-like material or adhesive, which is stretched or sized to cover the size and shape of the selected area of the body art application. The user may adjust the size of the cover thorough the use of a stretch release adhesive tape structure. Once the selected area of body art is covered, the user may then manually cut off or physically remove any excess tape structure so that only the area of body art is fully covered.

Once the desired size and shape of the skin area or periphery of the selected body art application is ascertained, the cover or sheet like material of the present invention may also be affixed, attached or secured to the user's body, extremities, appendages, or other portion of the human torso through the use of fastening means, such as Velcro, adhesive or at least one plastic zip tie device which can be adjusted and sized to fit around the selected area of skin where the body art is applied. It is also contemplated that the present invention may also be utilized to cover and protect burns and other wounds.

The preferred embodiment of the present invention has been disclosed. A person of ordinary skill in the art would realize however, that certain modifications would come within the teachings of this invention. Therefore, the following claims should be studied to determine the true scope and content of the invention.

What is claimed is:

1. A system adapted to protect a newly applied body art, the system comprising:
a cover defined by:
a peripheral portion having a mating surface for placement on a skin surface, wherein the mating surface is shaped to surround newly applied body art on the skin surface,
a central portion joining the peripheral portion, wherein the central portion has an outer surface and an interior recessed surface; and
a retention member adapted to affix the cover to the skin,
wherein placement and affixing of the cover around and over the newly applied body art creates a gapped region between the central portion and the newly applied body art and substantially prevents destructive contact to the same.

2. The system of claim 1, wherein the body art is selected from a tattoo, piercing, scaring, insertion of foreign objects, removal of skin, or a combination thereof.

3. The system of claim 1, wherein the cover is flexible.

4. The system of claim 1, wherein the cover comprises a metal.

5. The system of claim 1, wherein the cover comprises a plastic.

6. The system of claim 1, wherein the central portion defines one or more perforations adapted to allow air circulation to the newly applied body art or to allow the application of agents for facilitating in the healing process or both.

7. The system of claim 1, wherein placement of the cover onto the surface of the skin creates a substantially waterproof environment in the gapped region thereby substantially preventing the ingress or egress of water.

8. The system of claim 1, wherein the cover comprises of an opaque material, which substantially prevents exposure of the skin having the body art to sunlight.

9. The system of claim 1, wherein the retention member is selected from tape, Velcro, elastic, bandages, gels, adhesives, gauzes, wraps or combinations thereof.

10. The system of claim 8, wherein the retention member further acts to create a substantially waterproof environment in the gapped region.

11. A method of protecting a newly applied tattoo comprising the steps of:
providing a skin surface having a newly applied tattoo;
placing a cover over the newly applied body art, the cover defined by:
a peripheral portion having a mating surface for placement on a skin surface, wherein the mating surface is shaped to surround newly applied body art on the skin surface,
a central portion joining the peripheral portion, wherein the central portion has an outer surface and an interior recessed surface;
affixing the cover to the skin using a retention member, wherein a gapped region is created between the central portion and the newly applied body art, which substantially prevents destructive contact to the newly applied body art.

12. The method of claim 10, further comprising the step of applying an agent to the newly applied body art to facilitate in the healing of the same.

13. The method of claim 10, wherein the central portion defines one or more perforations adapted to allow air circulation to the newly applied body art or to allow the application of agents for facilitating in the healing process or both.

14. The method of claim 10, wherein placement of the cover onto the surface of the skin creates a substantially waterproof environment in the gapped region thereby substantially preventing the ingress or egress of water.

15. The method of claim 10, wherein the retention member is selected from tape, Velcro, elastic, bandages, gels, adhesives, gauzes, wraps or combinations thereof.

16. The method of claim 10, wherein the retention member further acts to create a substantially waterproof environment in the gapped region.

17. A kit for protecting a newly applied body art, the kit comprising:
a dome shield adapted to cover at least a portion of a newly applied body art on a skin surface, the dome shield having a recessed interior surface; and
a retention member for affixing the shield to a skin portion.

18. The kit of claim 17, further comprising an agent adapted to facilitate in the healing process of the newly applied body art.

19. The kit of claim 17, wherein the retention member is selected from tape, Velcro, elastic, bandages, gels, adhesives, gauzes, wraps or combinations thereof.

20. The kit of claim 17, further comprising additional wound protecting devices.
